(19) United States
(12) Patent Application Publication
(10) Pub. No.: US 2021/0353595 A1
(43) Pub. Date: Nov. 18, 2021

(54) USE OF MELATONIN IN PREPARATION OF MEDICAMENT FOR INHIBITING AND/OR KILLING BACTERIUM

(71) Applicant: South China Agricultural University, Guangdong (CN)

(72) Inventors: Wenkai REN, Guangdong (CN); Fang HE, Guangdong (CN); Yuanyi PENG, Guangdong (CN)

(21) Appl. No.: 16/996,887

(22) Filed: Aug. 18, 2020

(30) Foreign Application Priority Data
May 18, 2020 (CN) 202010418358.1

(51) Int. Cl. A61K 31/4045; A61P 31/04

(52) U.S. Cl. CPC A61K 31/4045; A61P 31/04

(57) ABSTRACT

The present invention provides for use of melatonin in the preparation of a medicament for inhibiting and/or killing a bacterium, as well as related compositions, and relates to the technical field of medicines. Methods of prevention and treatment of pathogenic bacteria infection disease states are also provided that leverage the remarkable bacteriostatic or bactericidal effects of melatonin, which damages the metabolic flux of a bacterium by specifically targeting a citrate synthase in a gram-negative bacterium. In an in vivo infection model of Pasteurella multocida, melatonin effectively reduces bacterial colonization in the lung and improves survival rate of mice.

### PRIORITY

This application is related to, and claims priority benefit of, Chinese Patent Application Serial No. 202010418358.1, filed May 18, 2020. The content of the aforementioned priority application is hereby expressly incorporated by reference in its entirety into this disclosure.

### TECHNICAL FIELD

The present invention relates to the technical field of medicines, and in particularly to use of melatonin in preparation of a medicament for inhibiting and/or killing a bacterium.

### STATEMENT OF SEQUENCE LISTING

A written Sequence Listing for the sequences described herein is appended hereto and the same Sequence Listing is provided in computer-readable form (CRF) encoded in a file that is submitted concurrently herewith and incorporated herein by reference. Such file, generated on Aug. 18, 2020, is entitled “ST25.txt”. The content of the CRF is the same, and the information recorded in the CRF is identical to, the written Sequence Listing provided herein, pursuant to 37 C.F.R. § 1.821(f).

### BACKGROUND

Among human and animal diseases, infectious diseases caused by pathogenic bacteria are spread most widely, which seriously threatens the public health around the world. So far, the discovery and application of antibiotics undoubtedly play an important role in resisting bacterial infection. However, due to the overuse and abuse of antibiotics in the pharmaceutical industry or animal husbandry, the emergence and prevalence of antibiotic resistance in human beings, animals and ecological environment are caused irreversibly. Alarmingly, plasmid-mediated interspecific and interspecific horizontal transfer of an antibiotic resistance gene accelerates the rapid spread of a resistance genetic element. Furthermore, residual antibiotics in veterinary drugs, animal products and the like products will also lead to serious environmental pollution, all constituting a great threat to human health. Therefore, there is an urgent need for more environmentally friendly, safer and more effective compounds to treat bacterial infectious diseases.

For bacterial infectious diseases, especially diseases caused by gram-negative bacterial infection, therapeutic drugs are very limited. The main reasons include: 1) the outer layer of the cell membrane of the gram-negative bacterium is composed of lipopolysaccharide, and can serve as an osmotic barrier to prevent antibiotics from entering the interior of the bacterium; and 2) the gram-negative bacterium has acquired multidrug resistance. Therefore, finding a safe and effective compound to inhibit the gram-negative bacterium is still an urgent problem to be solved in the current medical field.

Melatonin (N-acetyl-5-methoxytryptamine) is a natural compound, mainly a hormone secreted from the pineal gland of a vertebrate. Current studies have found that melatonin is widely used in sleep promoters by inhibiting orexin neurons in hypothalamus that interact with MT1 receptors. However, so far there has been no report on researches that melatonin inhibits bacteria and bacterial infectious diseases.

### SUMMARY

To address the aforementioned technical problem, the present invention provides use of safe and nontoxic melatonin in preparation of a medicament for inhibiting and/or killing a bacterium, and in particular melatonin can effectively inhibit and/or kill a gram-negative bacterium.

In order to realize the aforementioned objective of the present invention, the present invention provides the following technical solutions.

In at least one embodiment, a medicament for inhibiting and/or killing a bacterium is provided that comprises melatonin.

Preferably, the aforementioned bacterium is a gram-positive bacterium or a gram-negative bacterium.

Preferably, the gram-positive bacterium includes Bacillus cereus, Streptococcus pneumoniae, Pneumococcus, Listeria monocytogenes, Lactobacillus johnsonii or Staphylococcus aureus.

Preferably, the gram-negative bacterium includes Pasteurella multocida, Klebsiella pneumoniae, avian pathogenic Escherichia coli, Enterobacter sakazakii, Pseudomonas aeruginosa, Acinetobacter baumannii, Salmonella enteritidis, bovine meningitic Escherichia coli or Mannheimia haemolytica.

In certain embodiments, the present invention provides use of melatonin in preparation of a medicament that damages the cell membrane of a bacterium.

The present invention provides use of melatonin in preparation of a bacterial metabolic blocker composition.

Preferably, the pathways blocked by the aforementioned bacterial metabolic blocking composition may include, for example, pyruvate metabolism, citric acid cycle and tryptophan metabolism.

In other embodiments, the present invention provides use of melatonin in preparation of a medicament for inhibiting a citrate synthase activity in a gram-negative bacterium.

The present invention provides use of melatonin in preparation of a medicament for preventing and/or treating a bacterial infection or related disease.

The present invention provides use of melatonin in preparation of a medicament for inhibiting pulmonary inflammation.

Compared with the prior art, the technical solution of the present invention has the following beneficial effects.

The present invention provides novel uses of melatonin to inhibit and/or kill a bacterium for the first time, and in particular, is capable of inhibiting and/or killing a gram-negative bacterium. The bacteriostatic or bactericidal mechanism of melatonin indicates that melatonin damages the metabolic flux of a bacterium by specifically targeting citrate synthase in a gram-negative bacterium, resulting in membrane damage and release of cell contents. When administered to a host, compositions of the present disclosure comprising melatonin, and the methods of using the same in treatments, can also reduce the pathological changes of tissues and organs of the host, and inhibit the colonization of pathogenic bacteria in the host body, thereby effectively improving the survival of animals (including, without limitation, mammals) infected with pathogenic bacteria. In this manner, the compositions and methods hereof can be used to prevent and/or treat diseases caused by infection with pathogenic bacteria, and provide a basis for the development of new drugs. Furthermore, the natural bacteriostatic substance melatonin is harmless, pollution-free, safe and effective, and, when used as described herein, solves the problems of drug resistance, pollution and the like caused by antibiotic abuse.

### BRIEF DESCRIPTION OF THE DRAWINGS

In the figures, “CA” represents citric acid, and MT represents melatonin;

FIG. 1 is a graph showing the bacteriostatic activity of melatonin;

FIG. 2 is a graph showing the bacterial growth curve under a bactericidal concentration of melatonin according to at least one embodiment of the present disclosure;

FIG. 3 is a graph showing the bacterial count under a bactericidal concentration of melatonin according to at least one embodiment of the present disclosure;

FIG. 4 is a graph showing the melatonin-induced release of protein from bacterial cells;

FIG. 5 is a graph showing the melatonin-induced release of ATPase from bacterial cells;

FIG. 6 is a graph of the melatonin-induced release of alkaline phosphatase from bacterial cells;

FIG. 7 is a graph showing the melatonin-induced release of β-galactosidase from bacterial cells;

FIG. 8 is a graph showing the melatonin-induced release of reducing sugar from bacterial cells;

FIG. 9 is a graph showing that melatonin damages the cell membrane of a bacterium;

FIG. 10 is an SEM image of morphological changes of bacteria after treatment with melatonin;

FIG. 11 is a TEM image of morphological changes of bacteria after treatment with melatonin;

FIG. 12 is a diagram showing OPLS-DA scores of Pasteurella multocida in the presence and absence of melatonin, wherein D (●) represents a DMSO solvent control group and M (▪) represents melatonin;

FIG. 13 is a thermograph analysis diagram of metabolites that are significantly different after treatment with melatonin by non-targeted metabolomics;

FIG. 14 is a diagram of KEGG pathway enrichment analysis based on the significantly different metabolites after treatment with melatonin;

FIG. 15 is a diagram showing the activity of melatonin for significantly inhibiting the citrate synthase of Pasteurella multocida;

FIG. 16 is a graph showing that citric acid promotes the growth of Pasteurella multocida;

FIG. 17 is a line graph showing the effect of melatonin on the growth curve of Pasteurella multocida after the addition of citric acid, wherein CA represents citric acid;

FIG. 18 is a transmission electron microscopy (TEM) image of the morphology of Pasteurella multocida versus melatonin after the addition of citric acid, wherein CA represents citric acid;

FIG. 19 is a graph showing that citric acid significantly reduces the melatonin-induced release of reducing sugar, wherein CA represents citric acid;

FIG. 20 is a graph showing that citric acid significantly reduces the melatonin-induced release of protein, wherein CA represents citric acid;

FIG. 21 is a graph showing that citric acid significantly reduces the melatonin-induced release of alkaline phosphatase, wherein CA represents citric acid;

FIG. 22 is a graph showing that citric acid significantly reduces the melatonin-induced release of β-galactosidase, wherein CA represents citric acid;

FIG. 23 is a graph showing that citric acid significantly reduces the melatonin-induced release of ATPase, wherein CA represents citric acid;

FIG. 24 is a graph showing the detection of lactate dehydrogenase in mouse peritoneal primary macrophages by melatonin;

FIG. 25 is a graph showing the detection of lactate dehydrogenase in mouse lung epithelial cells by melatonin;

FIG. 26 is a graph showing the effect of melatonin on the enzymatic activity of citrate synthase of lung epithelial cells;

FIG. 27 is a graph showing the antibacterial activity of citric acid on gram-negative bacteria and gram-positive bacteria treated with melatonin, with subpart A showing data on Klebsiella pneumoniae, subpart B showing data on Pseudomonas aeruginosa, subpart C showing data on diplococcus pneumoniae, and subpart D showing data on streptococcus;

FIG. 28 is a graph showing the effect of citric acid on citrate synthase activity in gram-negative bacteria and gram-positive bacteria treated with melatonin;

FIG. 29 is a graph showing the survival rate of mice treated with melatonin before and after infection with Pasteurella multocida;

FIG. 30 is a graph showing the amount of colonization in the lung tissues of mice treated with melatonin before and after infection with Pasteurella multocida;

FIG. 31 is a diagram showing the preventive effect of melatonin on mice infected with bacteria;

FIG. 32 is a graph showing the effect of melatonin on the amount of colonization in the lung tissues of mice infected with bacteria;

FIG. 33 is a diagram showing the preventive effect on bacterial infection when melatonin is not injected any more 24 hours before infection with Pasteurella multocida;

FIG. 34 is a diagram showing the colonization amount in the lung tissues of the mice infected with the bacteria when melatonin is not injected any more 24 hours before infection with Pasteurella multocida;

FIG. 35 is a diagram showing the therapeutic effect of melatonin treatment on mice after infection with Pasteurella multocida;

FIG. 36 is a diagram showing the effect of melatonin treatment on the amount of colonization in the lung tissues of the mice after infection with Pasteurella multocida;

FIG. 37 is a graph showing the effect of melatonin on the expression amount of IL-10 as detected by RT-PCR after infection with Pasteurella multocida;

FIG. 38 is a graph showing the effect of melatonin on the expression amount of IL-6 as detected by RT-PCR after infection with Pasteurella multocida;

FIG. 39 is a graph showing the effect of melatonin on the expression amount of IFN-γ as detected by RT-PCR after infection with Pasteurella multocida;

FIG. 40 is a graph showing the effect of melatonin on the expression amount of TNF-α as detected by RT-PCR after infection with Pasteurella multocida;

FIG. 41 is a graph showing the effect of melatonin on the expression amount of IL-4 as detected by RT-PCR after infection with Pasteurella multocida;

FIG. 42 is a graph showing the effect of melatonin on the expression amount of IL-10 as detected by RT-PCR after infection with Pasteurella multocida;

FIG. 43 is a graph showing the effect of melatonin on the IL-10 content in the lung tissues of the mice as detected by an ELISA kit after infection with Pasteurella multocida;

FIG. 44 is a graph showing the effect of melatonin on the IL-6 content in the lung tissues of the mice as detected by an ELISA kit after infection with Pasteurella multocida;

FIG. 45 is a graph showing the effect of melatonin on the IFN-γ content in the lung tissues of the mice as detected by a ELISA kit after infection with Pasteurella multocida;

FIG. 46 is a graph showing the effect of melatonin on the TNF-α content in the lung tissues of the mice as detected by a ELISA kit after infection with Pasteurella multocida;

FIG. 47 is a graph showing the effect of melatonin on the IL-4 content in the lung tissues of the mice as detected by a ELISA kit after infection with Pasteurella multocida;

FIG. 48 is a graph showing the effect of melatonin on the IL-10 content in the lung tissues of the mice as detected by a ELISA kit after infection with Pasteurella multocida;

FIG. 49 is a graph showing the effect of melatonin on the IL-10 content in the serum of the mice as detected by a ELISA kit after infection with Pasteurella multocida;

FIG. 50 is a graph showing the effect of melatonin on the IL-6 content in the serum of the mice as detected by a ELISA kit after infection with Pasteurella multocida;

FIG. 51 is a graph showing the effect of melatonin on the IFN-γ content in the serum of the mice as detected by a ELISA kit after infection with Pasteurella multocida;

FIG. 52 is a graph showing the effect of melatonin on the TNF-α content in the serum of the mice as detected by a ELISA kit after infection with Pasteurella multocida;

FIG. 53 is a graph showing the effect of melatonin on the IL-4 content in the serum of the mice as detected by a ELISA kit after infection with Pasteurella multocida;

FIG. 54 is a graph showing the effect of melatonin on the IL-10 content in the serum of the mice as detected by a ELISA kit after infection with Pasteurella multocida; and

FIG. 55 is a diagram showing the pathway of melatonin in inhibiting and/or killing bacteria.

### DETAILED DESCRIPTION

The present invention provides a novel method for the use of melatonin in a preparation of a medicament for inhibiting and/or killing a bacterium.

In the present invention, the medicament for inhibiting and/or killing a bacterium preferably includes a compound or composition that comprises melatonin as an active ingredient and a pharmaceutically acceptable carrier. In the present invention, the content of melatonin in the medicament is preferably 1.5625 mg/mL. In the present invention, the pharmaceutically acceptable carrier for example includes one or more of a diluent, a colorant, a sweetener, a coating agent, an adhesive, an absorbent, a disintegrating agent, a releasing agent, a dispersing agent, a wetting agent, a cosolvent, a buffer and a surfactant. The dosage form of the medicament is preferably a solid, liquid or gas formulation, wherein the solid formulation is preferably powder, tablets, granules, pills, hard capsules, cream, ointment, plaster, gels, pastes, pulvis or patches. The liquid formulation is preferably a solution, a suspension, an injection, syrup, liniment, an emulsion, tincture, pulvis or a patch. The gas formulation is preferably an aerosol or spray. The medicament of the present invention can be administered orally, rectally, intraperitoneally, subcutaneously, intramuscularly, intravenously and intranasally.

Preferably, the aforementioned bacterium is a gram-positive bacterium or a gram-negative bacterium.

Preferably, the aforementioned gram-positive bacterium includes Bacillus cereus, Streptococcus pneumoniae, Pneumococcus, Listeria monocytogenes, Lactobacillus johnsonii or Staphylococcus aureus.

Preferably, the gram-negative bacterium includes Pasteurella multocida, Klebsiella pneumoniae, avian pathogenic Escherichia coli, Enterobacter sakazakii, Pseudomonas aeruginosa, Acinetobacter baumannii, Salmonella enteritidis, bovine meningitic Escherichia coli or Mannheimia haemolytica.

The present invention provides use of melatonin in preparation of a medicament that damages the cell membrane of a bacterium.

In the present invention, the medicament for inhibiting and/or killing a bacterium preferably includes melatonin as an active ingredient and a pharmaceutically acceptable carrier. In the present invention, the content of melatonin in the medicament is preferably 1.5625 mg/mL. In the present invention, the pharmaceutically acceptable carrier for example includes one or more of a diluent, a colorant, a sweetener, a coating agent, an adhesive, an absorbent, a disintegrating agent, a releasing agent, a dispersing agent, a wetting agent, a cosolvent, a buffer and a surfactant. The dosage form of the medicament is preferably a solid, liquid or gas formulation, wherein the solid formulation is preferably powder, tablets, granules, pills, hard capsules, cream, ointment, plaster, gels, pastes, pulvis or patches. The liquid formulation is preferably a solution, a suspension, an injection, syrup, liniment, an emulsion, tincture, pulvis or a patch. The gas formulation is preferably an aerosol or spray. The medicament of the present invention can be administered orally, rectally, intraperitoneally, subcutaneously, intramuscularly, intravenously and intranasally. The present invention provides use of melatonin in preparation of a bacterial metabolic blocker. In the present invention, the medicament for inhibiting and/or killing a bacterium preferably includes melatonin as an active ingredient and a pharmaceutically acceptable carrier. In the present invention, the content of melatonin in the medicament is preferably 1.5625 mg/mL. In the present invention, the pharmaceutically acceptable carrier for example includes one or more of a diluent, a colorant, a sweetener, a coating agent, an adhesive, an absorbent, a disintegrating agent, a releasing agent, a dispersing agent, a wetting agent, a cosolvent, a buffer and a surfactant. The dosage form of the medicament is preferably a solid, liquid or gas formulation, wherein the solid formulation is preferably powder, tablets, granules, pills, hard capsules, cream, ointment, plaster, gels, pastes, pulvis or patches. The liquid formulation is preferably a solution, a suspension, an injection, syrup, liniment, an emulsion, tincture, pulvis or a patch. The gas formulation is preferably an aerosol or spray. The medicament of the present invention can be administered orally, rectally, intraperitoneally, subcutaneously, intramuscularly, intravenously and intranasally. Preferably, the pathways of the aforementioned bacterial metabolism blocking include pyruvate metabolism, citric acid cycle and tryptophan metabolism.

The present invention provides use of melatonin in preparation of a medicament for inhibiting a citrate synthase activity in a gram-negative bacterium.

In the present invention, the medicament for inhibiting and/or killing a bacterium preferably includes melatonin as an active ingredient and a pharmaceutically acceptable carrier. In the present invention, the content of melatonin in the medicament is preferably 1.5625 mg/mL. In the present invention, the pharmaceutically acceptable carrier for example includes one or more of a diluent, a colorant, a sweetener, a coating agent, an adhesive, an absorbent, a disintegrating agent, a releasing agent, a dispersing agent, a wetting agent, a cosolvent, a buffer and a surfactant. The dosage form of the medicament is preferably a solid, liquid or gas formulation, wherein the solid formulation is preferably powder, tablets, granules, pills, hard capsules, cream, ointment, plaster, gels, pastes, pulvis or patches. The liquid formulation is preferably a solution, a suspension, an injection, syrup, liniment, an emulsion, tincture, pulvis or a patch. The gas formulation is preferably an aerosol or spray. The medicament of the present invention can be administered orally, rectally, intraperitoneally, subcutaneously, intramuscularly, intravenously and intranasally.

The present invention provides use of melatonin in preparation of a medicament for preventing and/or treating a bacterial infection disease.

In the present invention, the medicament for inhibiting and/or killing a bacterium preferably includes melatonin as an active ingredient and a pharmaceutically acceptable carrier. In the present invention, the content of melatonin in the medicament is preferably 1.5625 mg/mL. In the present invention, the pharmaceutically acceptable carrier for example includes one or more of a diluent, a colorant, a sweetener, a coating agent, an adhesive, an absorbent, a disintegrating agent, a releasing agent, a dispersing agent, a wetting agent, a cosolvent, a buffer and a surfactant. The dosage form of the medicament is preferably a solid, liquid or gas formulation, wherein the solid formulation is preferably powder, tablets, granules, pills, hard capsules, cream, ointment, plaster, gels, pastes, pulvis or patches. The liquid formulation is preferably a solution, a suspension, an injection, syrup, liniment, an emulsion, tincture, pulvis or a patch. The gas formulation is preferably an aerosol or spray. The medicament of the present invention can be administered orally, rectally, intraperitoneally, subcutaneously, intramuscularly, intravenously and intranasally. The present invention provides use of melatonin in preparation of a medicament for inhibiting pulmonary inflammation.

In the present invention, the medicament for inhibiting and/or killing a bacterium preferably includes melatonin as an active ingredient and a pharmaceutically acceptable carrier. In the present invention, the content of melatonin in the medicament is preferably 1.5625 mg/mL. In the present invention, the pharmaceutically acceptable carrier for example includes one or more of a diluent, a colorant, a sweetener, a coating agent, an adhesive, an absorbent, a disintegrating agent, a releasing agent, a dispersing agent, a wetting agent, a cosolvent, a buffer and a surfactant. The dosage form of the medicament is preferably a solid, liquid or gas formulation, wherein the solid formulation is preferably powder, tablets, granules, pills, hard capsules, cream, ointment, plaster, gels, pastes, pulvis or patches. The liquid formulation is preferably a solution, a suspension, an injection, syrup, liniment, an emulsion, tincture, pulvis or a patch. The gas formulation is preferably an aerosol or spray. The medicament of the present invention can be administered orally, rectally, intraperitoneally, subcutaneously, intramuscularly, intravenously and intranasally.

Experimental Materials

1. Strains Mainly Used in Examples

The present invention has no special limitation on the source of the strains, and it will be appreciated that any strain well known to those skilled in the art can be used.

2. Materials Mainly Used in the Examples

For the examples provided herein, melatonin is available from Sangon Biotech under the product code A600605. O-nitrophenyl-β-D-galactopyranoside is available from Sangon Biotech under the product code A602361.

The kit for protein detection is available from Beyotime under the product code P0006C.

The alkaline phosphatase kit is available from Nanjing Jiancheng Bioengineering Institute under the product code A059-1-1.

The reducing sugar kit is available from Solarbio under the product code BCO230.

The LIVE/DEAD BacLight bacterial detection kit is available from Invitrogen under the product code L7012.

The kit used for determining the citrate synthase activity is available from Sigma under the product code MAK193.

The LDH detection kit is available from Beyotime under the product code C0017.

The mice used in the examples of the present disclosure were female C57BL/6 mice purchased from Experimental Animal Center, Third Military Medical University, Chongqing, China. The mice weighed 18-22 g and were 6-8 weeks old. They were raised in individual ventilated cages without pathogens (at a temperature of 20-30° C., relative humidity of 50-60%, and an illumination period of 12 hours every day), and had free access to feedstuff and water.

In the present disclosure, analysis was conducted by a statistical method adopting one-way analysis of variance, and is expressed herein as an average±SD, *P<0.05, **P<0.01, ***P<0.001, ****P<0.0001, wherein P<0.05 refers to the difference being statistically significant.

In the present disclosure, the raw constituents mentioned are all commercially available products well known to those skilled in the art, unless otherwise specified.

The technical solutions in the present invention will be clearly and completely described below in conjunction with the embodiments of the present invention. The described embodiments are merely provided as explanatory examples, are not intended to be limiting, and indeed do not comprise all of the embodiments of the present invention. All other embodiments obtained by a person of ordinary skill in the art based on the embodiments of the present invention without creative efforts shall fall within the protection scope of the present invention.

### EXAMPLE 1

### Broad-Spectrum Bacteriostatic and/or Bactericidal Test of Melatonin

1 mL of a fresh suspension of bacteria (1×10⁹ CFU) at the logarithmic growth stage was added into 100 mL containing different concentrations (50 mg/mL, 25 mg/mL, 12.5 mg/mL, 6.25 mg/mL, 3.125 mg/mL, 1.5625 mg/mL, 0.78125 mg/mL, 0.390625 mg/mL) of melatonin. A melatonin (MT)-free solution diluted with DMSO was used as a control group. The samples were shaken at 37° C. and a rotation speed of 120 r/min with protection from light for 12 hours, and then subjected to detection of the minimal inhibitory concentration (MIC) of MT on bacteria. Then, 0.1 mL of the aforementioned transparent bacterial solution was pipetted onto a sterilized agar plate of a specific culture medium and coated evenly, incubated in an incubator at 37° C. for 24 hours, and then subjected to detection of the minimum bactericidal concentration (MBC) of MT on pathogens, wherein the minimum concentration of MT at which no bacterium grew was the MBC of MT on pathogens. Each experiment was carried out for 3 times, and each group was set in triplicate.

The MIC and MBC results of melatonin on gram-negative bacteria and gram-positive bacteria are shown in Table 1:

TABLE 1
MIC and MBC results of melatonin on gram-
negative and gram-positive bacteria

| | MIC (mg/mL) | MBC (mg/mL) |
|---|---|---|
| Species of gram-negative bacteria | | |
| Pasteurella multocida | 1.5625 | 3.125 |
| Klebsiella pneumoniae | 3.125 | 6.25 |
| Avianpathogenic Escherichia coli | 6.25 | 12.5 |
| Enterobacter sakazakii | 6.25 | 25 |
| Pseudomonas aeruginosa | 12.5 | 12.5 |
| Acinetobacter baumannii | 12.5 | 12.5 |
| Salmonella enteritidis (ATCC85090) | 12.5 | 12.5 |
| Bovine meningitic Escherichia coli | 12.5 | 12.5 |
| Escherichia coli C83902 (ATCC72864) | 12.5 | 25 |
| Mannheimia haemolytica | 12.5 | 25 |
| Species of gram-positive bacteria | | |
| Bacillus cereus | 6.25 | 12.5 |
| Streptococcus pneumoniae | 12.5 | 25 |
| Pneumococcus | 12.5 | 25 |
| Listeria monocytogenes (ATCC53005) | 12.5 | 50 |
| Lactobacillus johnsonii | 25 | 50 |
| Staphylococcus aureus (ATCC25923) | 50 | 50 |

It can be seen from Table 1 that melatonin exhibited a bacteriostatic or bactericidal activity against both gram-negative bacteria and gram-positive bacteria, with MIC values ranging from 1.5625 to 50 mg/mL and MBC values ranging from 3.125 to 50 mg/mL. Notably, melatonin showed the most effective antibacterial or bactericidal activity against Pasteurella multocida, with MIC and MBC of 1.5625 mg/mL and 3.125 mg/mL respectively.

### EXAMPLE 2

### Test of Bacteriostatic Activity of Melatonin Against Bacteria

In order to further verify the bacteriostatic activity of melatonin against Pasteurella multocida, the bacterial growth curve was determined in view of an increase of melatonin concentration. Here, 1 mL of Pasteurella multocida (1.0×10⁹ CFU) at the logarithmic growth stage was added into 100 mL of fresh and sterile Martin broth medium containing a MT concentration lower than MBC (1 mg/mL, 1.5 mg/mL and 2 mg/mL), and melatonin-free DMSO was used as a solvent control group. Then the bacteria were cultured in a shaker at a constant temperature of 37° C. at 220 rpm, and the bacterial culture was determined for OD600 every 2 hours with an ultraviolet spectrophotometer to detect the effect of melatonin on the bacterial growth curve. Each experiment was carried out for three times, and each group was set in triplicate.

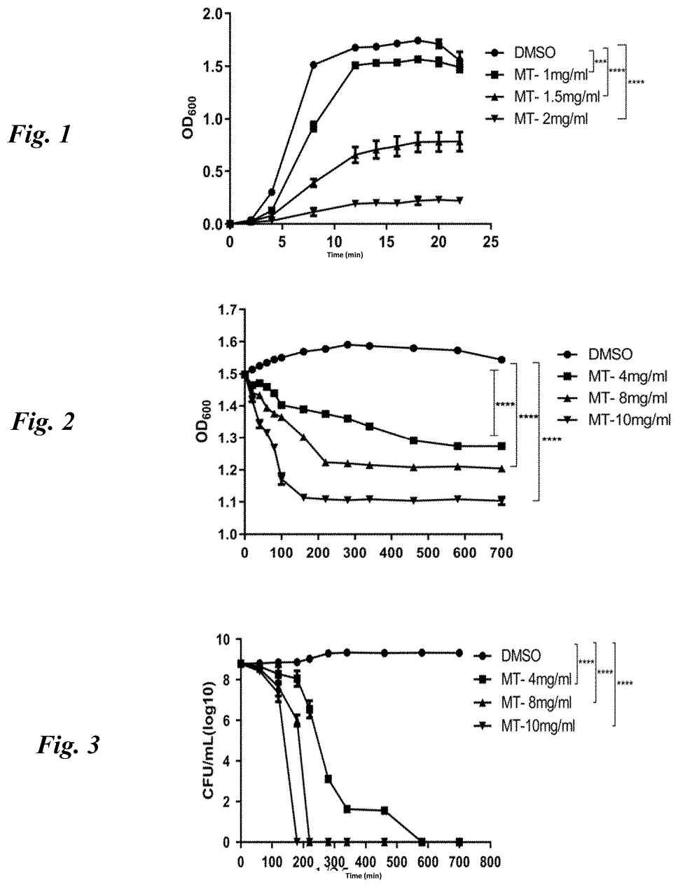

The results shown in FIG. 1 illustrate that melatonin significantly inhibits bacterial growth.

### EXAMPLE 3

### Killing Bacteria by Melatonin

Into 100 mL of fresh and sterile Martin broth medium, 1 mL of Pasteurella multocida (1×10⁹ CFU) at the logarithmic growth stage was added. The bacteria were cultured in a constant-temperature shaker under conditions of 37° C. and 220 rpm for 8 hours. Subsequently, MT of different concentrations (4 mg/mL, 8 mg/mL and 10 mg/mL) higher than the MBC were added into the bacterial culture, while the MT-free DMSO was used as the solvent control group. After the addition of MT, the bacterial culture was continuously measured for OD600 every 20 minutes to depict a growth curve under the action of melatonin at a concentration higher than the MBC. At the same time, 100 μL of the bacterial culture was evenly coated on a Martin agar plate, and incubated at 37° C. for 24 h so as to calculate the number of living bacteria for bacterial counting.

The results of these studies are shown in FIGS. 2 and 3. While it took 10 hours for 4 mg/mL of melatonin to kill all bacteria, it took only 4 hours for a higher concentration of melatonin to kill all bacteria. Therefore, as supported by the data shown in FIGS. 2 and 3, melatonin at a concentration higher than the MBC showed a significant bactericidal effect in a dose-dependent manner.

### EXAMPLE 4

### Leakage of Bacterial Cell Contents Caused by Melatonin

1 mL of Pasteurella multocida (1×10⁹ CFU) at the logarithmic growth stage was added into 100 mL of fresh and sterile Martin broth medium containing different concentrations (1 mg/mL, 2 mg/mL, 3 mg/mL, 4 mg/mL, 6 mg/mL, 8 mg/mL and 10 mg/mL) of MT. Melatonin-free DMSO was used as a solvent control group, ampicillin (100 μg/mL) was used as a positive control group, and bacterial cells were cultured in a shaker at 37° C. and 220 rpm for 12 h. Thereafter, 1 mL of the bacterial solution was taken and centrifuged at 3000 r/min for 10 minutes, and the supernatant and precipitated cells were collected, respectively. At the same time, 2.5 mmol/mL of o-nitrophenyl-β-D-galactopyranoside (ONPG) and Pasteurella multocida were added to detect β-galactosidase. The concentrations of protein, alkaline phosphatase (AKP) and reducing sugar in the supernatant were determined sequentially using respective determination kits. The β-galactosidase in the supernatant was determined by measuring the optical density at 420 nm (OD420). Each experiment was carried out for three times, and each group was set in triplicate.

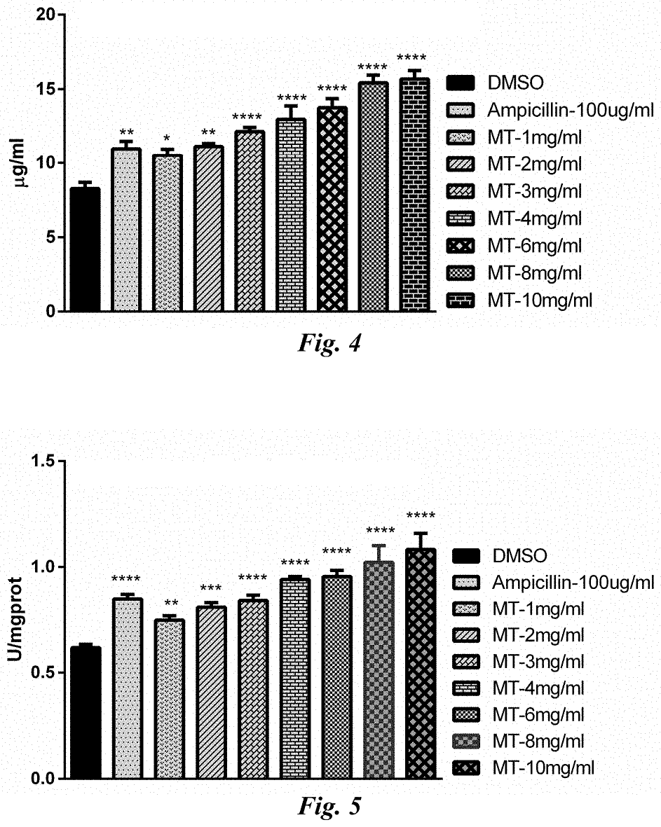

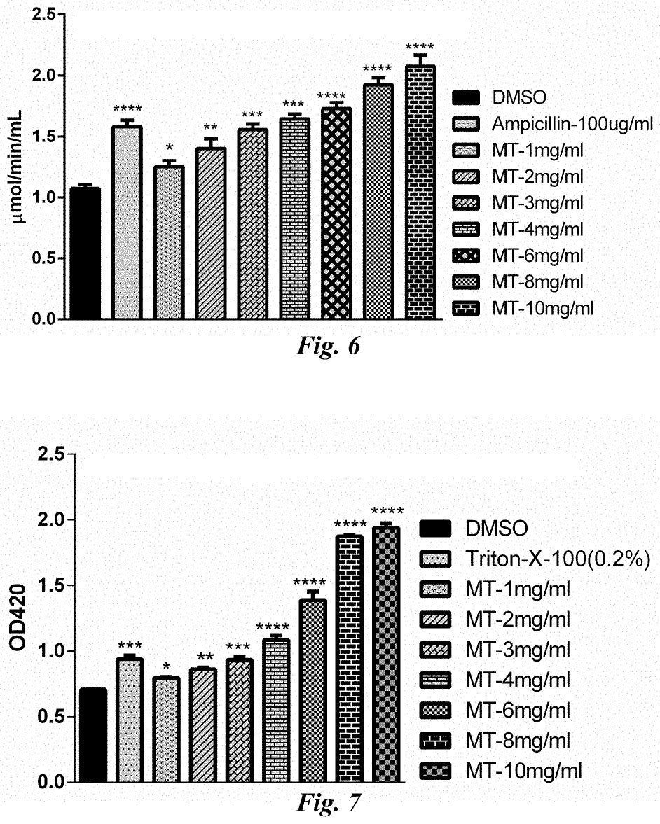

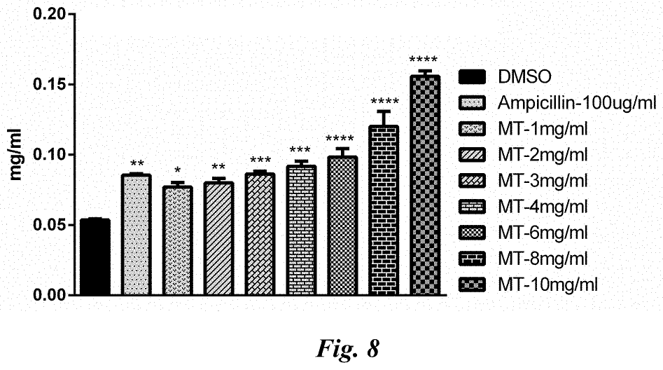

The results of these studies are shown in FIGS. 4-8. It can be seen from the results that melatonin led to the increase in the contents of extracellular proteins, reducing sugar and alkaline phosphatase (which is located between the cell wall and the cell membrane) and the increase in the activities of extracellular β-galactosidase (which is located within the cell) and ATPase. These results indicate that melatonin damages the integrity of the cell membrane, leading to the leakage of cell contents.

### EXAMPLE 5

### Destruction of Bacterial Cell Membrane Caused by Melatonin

The integrity of bacterial cell membrane was evaluated with a LIVE/DEAD BacLight bacterial detection kit. 1 mL of bacterial culture was centrifuged at 3000 rpm for 10 minutes, the supernatant was removed, and the cells were washed with 10 mL of 0.85% NaCl for 3 times. The precipitated cells were resuspended and mixed with 1 mL of 0.85% NaCl. Then a mixture of SYTO9 fluorescent nucleic acid dye and propidium iodide mixed at the equal volume (3 μL each) was added into the resuspended solution. The mixture was thoroughly mixed and incubated in the dark at room temperature for 15 minutes. 5 μL of suspension of stained bacteria was added between a slide glass and a 18 mm square cover glass. (The combination of two nucleic acid dyes in this kit can distinguish living bacteria with intact membranes from dead bacteria with damaged membranes.) The living bacteria were dyed green with SYTO9, while the dead bacteria presented as red because of the interaction between propidium iodide and the double-stranded DNA of the bacteria. The fluorescence micrographs of stained bacteria were taken by OLYMPUS IX51 inverted microscope. Each experiment was carried out in triplicate for three times.

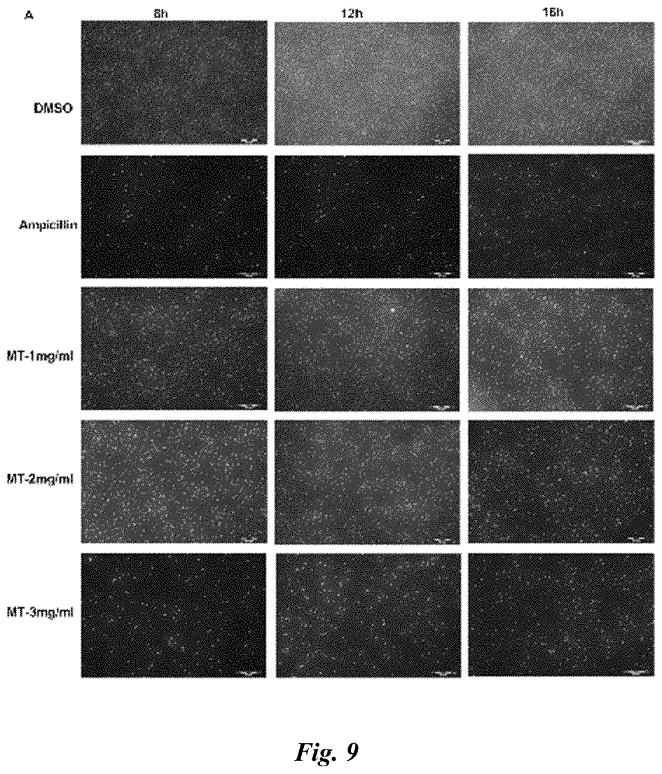

FIG. 9 shows that in the DMSO control group, only a small amount of red fluorescence appeared at 16 hours, but no red fluorescence appeared at 8 or 12 hours. In contrast, for those groups where the amount of melatonin was greater than the MIC concentration, the red fluorescence increased and the green fluorescence decreased, which was time-dependent and dose-dependent. Therefore, these results support that the damage caused by melatonin to the bacterial cell membrane is time-dependent and dose-dependent.

### EXAMPLE 6

### Scanning Electron Microscope (SEM) and Transmission Electron Microscope (TEM) Images of Integrity of Bacterial Cell Membrane as Damaged by Melatonin

The effect of melatonin on the morphologies of P. multocida cells were evaluated employing SEM and TEM. 1 mL of fresh Pasteurella multocida (1×10⁹ CFU) at the logarithmic growth stage was added into 100 mL of fresh Martin liquid medium containing different concentrations (1, 2 or 3 mg/mL) of MT. MT-free DMSO was used as a solvent control group and ampicillin (100 μg/mL) was used as a positive control group. All cultures were shaken at 220 rpm in a shaker at 37° C. for 8 hours, 12 hours or 16 hours. The bacterial samples were centrifuged at 3000 r/min for 10 minutes, the supernatant was removed, and cells were collected and washed with PBS for 3 times. Then the cells were fixed with a fixation solution of 2.5% fresh glutaraldehyde for 6 hours, and washed with PBS for 3 times, each time for 10 minutes. Subsequently, the SEM samples were dehydrated with 50%, 70%, 80% and 90% ethanol for 15 minutes respectively, and then dehydrated with 100% ethanol for 3 times, each time for 30 minutes. Ethanol was removed by washing with tert-butyl alcohol for three times, each time for 30 minutes. The samples were dried by a freeze dryer and plated with 10 nm of gold film by an ion sputtering device. The samples were examined using a field scanning electron microscope (FESEM, Hitch SU8010).

The TEM samples were fixed with 1%-2% citric acid for 2-3 hours, and washed with phenyl-5-(4-diphenyl)-1,3,4-oxazole (PBD) 3 times, each time for 10 minutes. The samples were dehydrated with 30%, 50%, 70%, 80% and 90% ethanol for 20 minutes respectively, and then dehydrated with 100% ethanol 3 times, each time for 30 minutes. Ethanol was replaced with acetone three times, each time for 30 minutes. The bacterial samples were placed on a copper grid with a formvar membrane, and negatively stained with phosphotungstic acid (2% v/v, pH=6.7). The surface area of the sample was less than 0.2 mm×0.2 mm, and slice thickness was between 50 and 90 nm (Lycra UC7). The samples were detected by TEM (Hitachi H-7650) at 80 kV, and photographs of the samples were taken by a Gatan832 CCD camera (Gatan). Each experiment was carried out twice, and each group was set in triplicate.

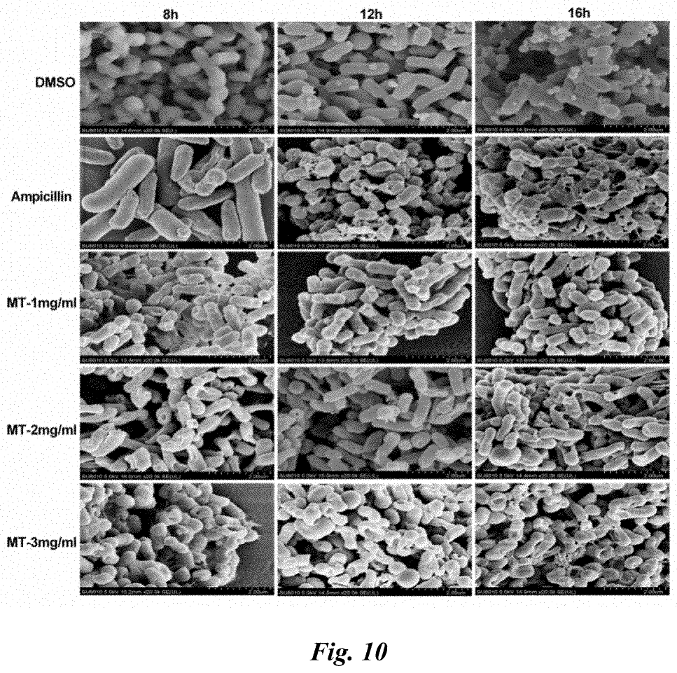

FIGS. 10 and 11 show that, before melatonin treatment, all bacterial cells showed intact outer membrane (OM) and cytoplasmic membrane (CM). Following treatment, the cells treated with melatonin showed many holes passing through the OM, peptidoglycan (PG) layers and CM in SEM analysis (see FIG. 10). Interestingly, in the TEM image (see FIG. 11), it was found that there was obvious cytoplasmic vacuolization in cells treated with melatonin, which may be caused by aggregation of intracellular proteins or leakage of cell contents.

Therefore, the results illustrated in FIGS. 4-11 show that melatonin killed bacteria by damaging bacterial membranes and inducing the leakage of cell contents.

### EXAMPLE 7

### Test of Metabonomic Analysis

1 mL of fresh Pasteurella multocida (P. multocida) at the logarithmic growth stage was added into 100 mL of fresh Martin liquid medium containing 1 mg/mL of MT, and MT-free DMSO was used as a solvent control group. All cultures were incubated with shaking at 37° C. and 220 rpm for 12 hours. The bacterial samples were centrifuged at a speed of 3000 rpm/min at 4° C. for 10 minutes, the supernatant was removed, and the bacterial cells were collected and washed with PBS for 3 times. The bacterial cells were rapidly frozen in liquid nitrogen, transferred to dry ice, and sent to Shanghai Baiqu Biomedical Technology Co., Ltd. for metabolomic analysis (Q Exactive Orbitrap, Thermo Fisher Scientific, USA). Through the non-targeted metabonomic study of Pasteurella multocida cells before and after melatonin treatment, it was clarified which metabolic pathway of the bacteria is affected by melatonin in exerting the antibacterial activity of melatonin.

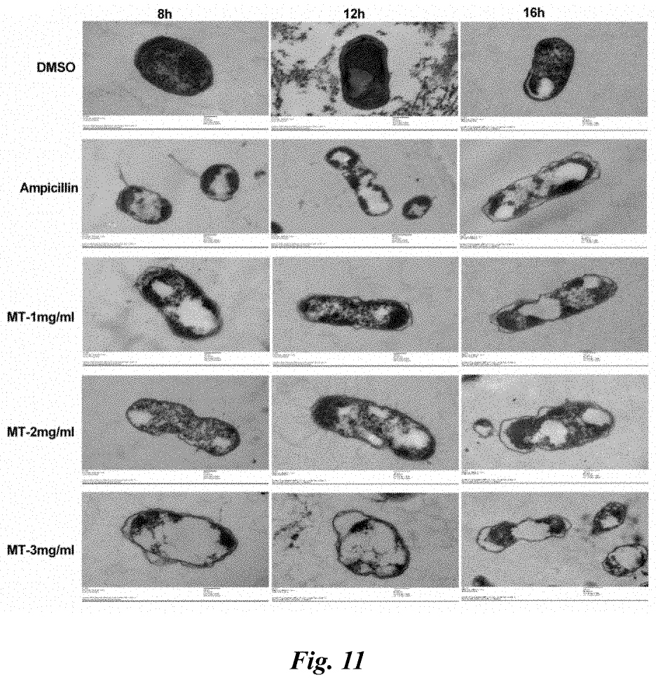

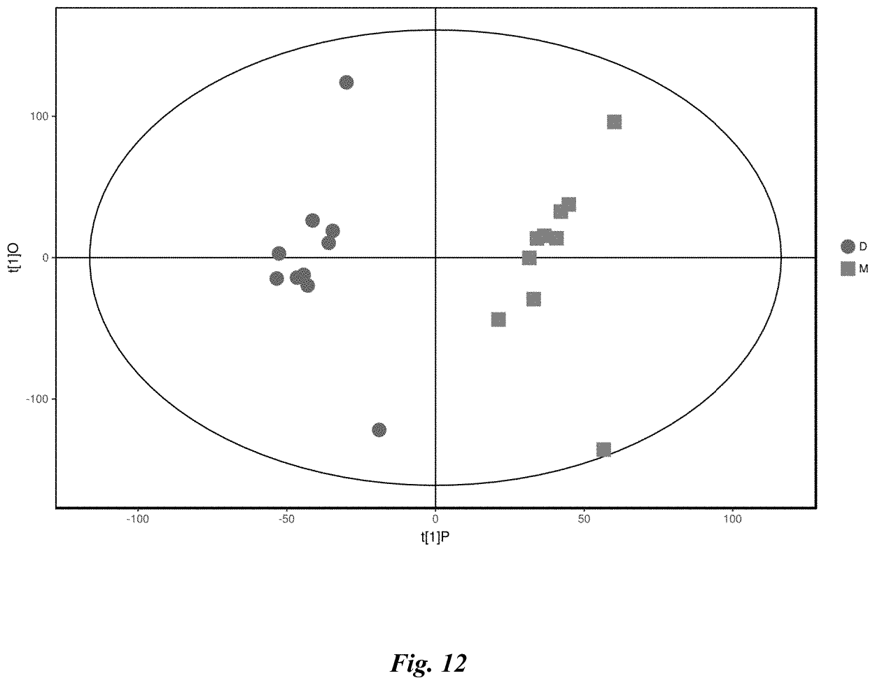

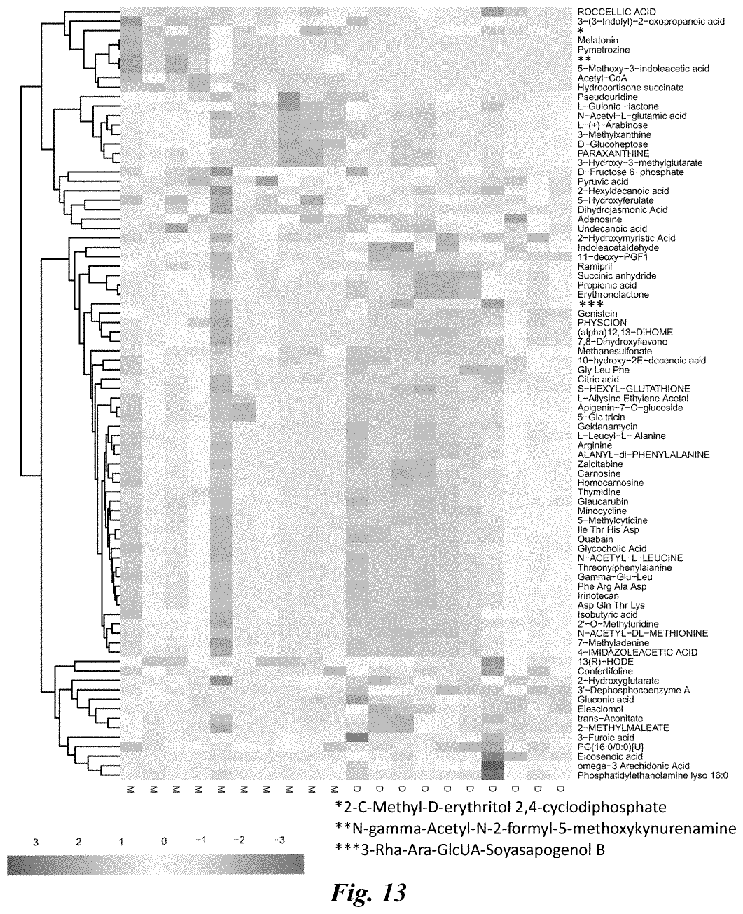

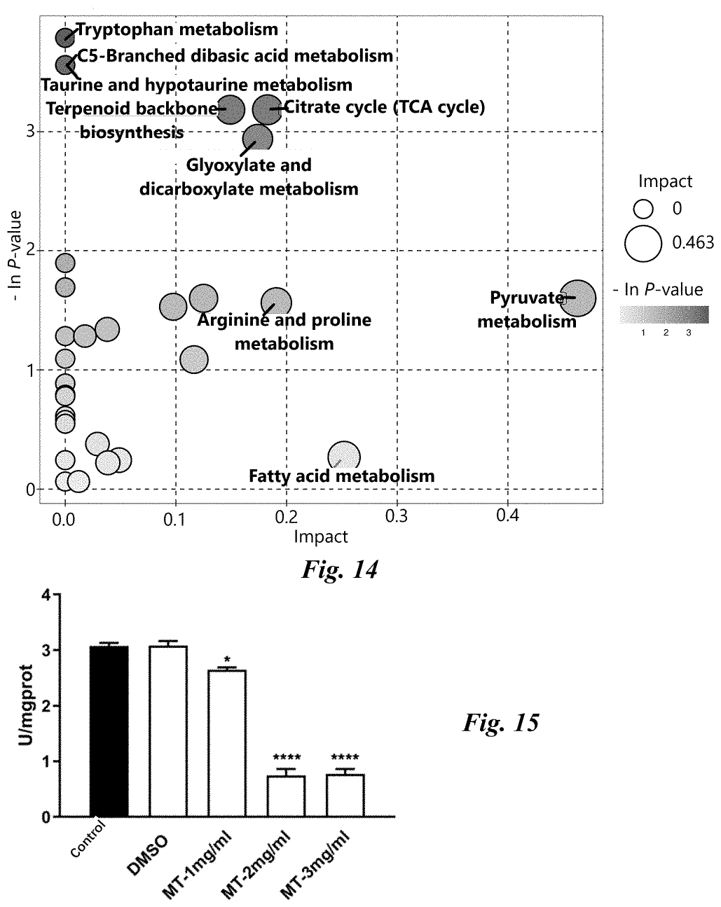

As shown in FIGS. 12 and 13, there were a large number of metabolites with significant differences after melatonin treatment. As seen in FIG. 14, these metabolites are mainly concentrated in pyruvic acid metabolism, citric acid cycle and tryptophan metabolism. Based on these findings, the changes of related metabolites in the TCA cycle were further intensively studied. Interestingly, it was found that pyruvic acid and acetyl coenzyme A were significantly up-regulated and citric acid was significantly down-regulated in bacterial cells treated with melatonin. The results show that melatonin blocked the pathway from acetyl coenzyme A to citric acid, resulting in the accumulation of pyruvic acid and acetyl coenzyme A.

### EXAMPLE 8

### Test of Inhibiting Citrate Synthase Activity of Pasteurella multocida by Melatonin

Based on the discovery of metabolomics, the enzyme activity of citrate synthase after melatonin treatment was studied. 1 mL of Pasteurella multocida (1×10⁹ CFU) at the logarithmic growth stage was added into 100 mL of fresh and sterile Martin broth medium containing different concentrations (1 mg/mL, 2 mg/mL, and 3 mg/mL) of MT. Melatonin-free DMSO was used as a solvent control group, and the bacterial cells were cultured in a constant-temperature shaker at 37° C. and 220 rpm for 12 h. Thereafter, 1 mL of the bacterial solution was centrifuged at 3000 r/min for 10 minutes, and the supernatant and precipitated cells were collected, respectively. The citrate synthase activity was determined using a determination kit according to the protocol of the instructions. Each experiment was repeated for three times, and each group was set in triplicate.

In order to explore whether the decrease of citric acid induced by melatonin is the key link of the bacteriostasis of melatonin, the influence of exogenous citric acid on the bacteriostatic effect of melatonin was also studied. Here, 1 mL of Pasteurella multocida (1×10⁹ CFU) at the logarithmic growth stage was added into 100 mL of fresh and sterile Martin broth medium containing different concentrations of citric acid (CA-1 mg/ml, CA-2 mg/mL, and CA-3 mg/mL), and citric acid-free DMSO was used as a solvent control group. The bacterial cells were cultured in a constant-temperature shaker at 37° C. and 220 rpm for 30 minutes. Then, the growth curve of the cells was determined. Each experiment was repeated for three times, and each group was set in triplicate.

Additionally, 1 mL of Pasteurella multocida (1×10⁹ CFU) at the logarithmic growth stage was added into 100 mL of fresh and sterile Martin broth medium containing different concentrations of melatonin and citric acid (CA) (MT-1 mg/mL, MT-2 mg/mL, MT-3 mg/mL, CA-1 mg/mL, CA-2 mg/mL, CA-3 mg/mL, MT-1 mg/mL-CA-1 mg/mL, MT-2 mg/mL-CA-2 mg/mL, MT-3 mg/mL-CA-3 mg/mL). The melatonin-free DMSO was used as a solvent control group, and the bacterial cells were cultured in a shaker at 37° C. and 220 rpm for 12 h. Thereafter, 1 mL of the bacterial solution was taken and centrifuged at 3000 r/min for 10 minutes, and the supernatant and precipitated cells were collected, respectively. At the same time, 2.5 mmol/mL of o-nitrophenyl-3-D-galactopyranoside (ONPG) and Pasteurella multocida were added to detect (3-galactosidase. The concentrations of protein, alkaline phosphatase (AKP) and reducing sugar in the supernatant were determined sequentially using respective determination kits. The β-galactosidase in the supernatant was determined by measuring the optical density at 420 nm (OD420). Each experiment was carried out for three times, and each group was set in triplicate.

1 mL of Pasteurella multocida (1×10⁹ CFU) at the logarithmic growth stage was also added into 100 mL of fresh and sterile Martin broth medium containing different concentrations of melatonin and citric acid (CA) (MT-1 mg/mL, MT-1 mg/mL-CA-1 mg/mL, MT-1 mg/mL-CA-2 mg/mL, MT-1 mg/mL-CA-3 mg/mL; MT-2 mg/mL, MT-2 mg/mL-CA-1 mg/mL, MT-2 mg/mL-CA-2 mg/mL, MT-2 mg/mL-CA-3 mg/mL; MT-3 mg/mL, MT-3mg/mL-CA-1 mg/mL, MT-3 mg/mL-CA-2 mg/mL, and MT-3 mg/mL-CA-3 mg/mL). DMSO not containing melatonin and citric acid was used as a solvent control group, and the bacterial cells were cultured in a constant-temperature shaker at 37° C. and 220 rpm for 12 h. Then the growth curve of the cells were determined. Each experiment was carried out for three times, and each group was set in triplicate.

Further, 1 mL of Pasteurella multocida (1×10⁹ CFU) at the logarithmic growth stage was added into 100 mL of fresh and sterile Martin broth medium containing different concentrations of melatonin and citric acid (MT-1 mg/mL, MT-2 mg/mL, MT-3 mg/mL, CA-1 mg/mL, CA-2 mg/mL, CA-3 mg/mL, MT-1 mg/mL-CA-1 mg/mL, MT-2 mg/mL-CA-2 mg/mL, MT-3 mg/mL-CA-3 mg/mL). The melatonin-free DMSO was used as a solvent control group, and the bacterial cells were cultured in a constant-temperature shaker at 37° C. and 220 rpm for 12 h. Thereafter, 1 mL of the bacterial solution was centrifuged at 3000 r/min for 10 minutes, and the supernatant and precipitated cells were collected, respectively. The citrate synthase activity was determined using a determination kit according to the protocol of the instructions. Each experiment was repeated for three times, and each group was set in triplicate.

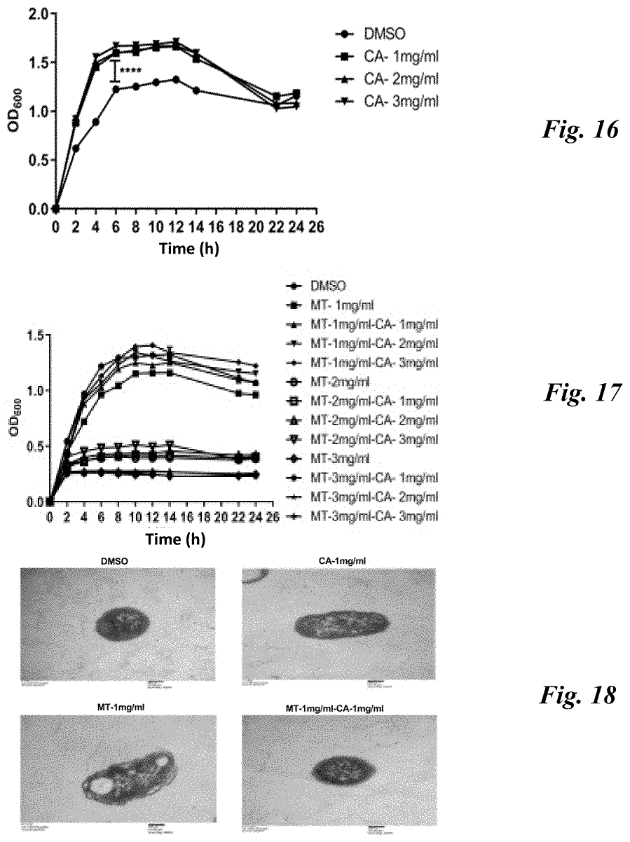

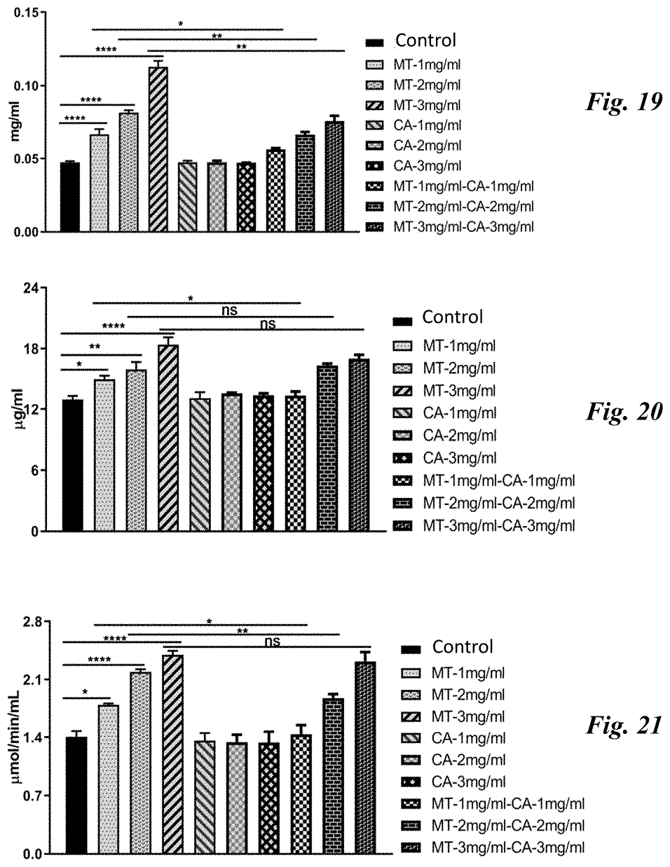

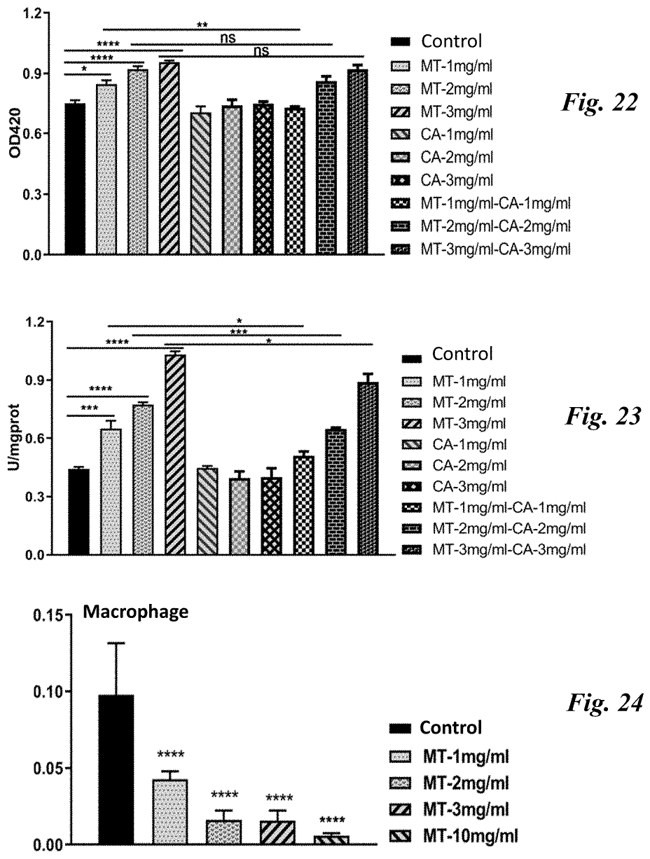

The results shown in FIG. 15 illustrate that melatonin significantly reduced the activity of citrate synthase in the bacteria and the culture medium. It can be seen from FIG. 16 that the addition of citric acid promoted the growth of Pasteurella multocida, indicating that citric acid was an important metabolite for normal growth of bacteria. The results shown in FIG. 17 show that the addition of citric acid reduces the antibacterial activity of melatonin against Pasteurella multocida. The TEM analysis also shows (see FIG. 18) that under the combined action of melatonin and citric acid, the cytoplasmic vacuolization and irregular cell membrane of the bacteria only caused by melatonin disappeared. Furthermore, the data shown in FIGS. 19-23 indicates that, citric acid significantly reduced the release of reducing sugar, proteins, AKP enzymes and β-galactosidase and the activity of ATPase induced by melatonin, indicating that citric acid antagonized the damage of melatonin to the bacterial cell membrane.

### EXAMPLE 9

### Test of Effect of Melatonin on Citrate Synthase in Mammals and Other Bacteria

In view of the important role of citrate synthase in mammalian cells, the cytotoxicity of melatonin in mouse peritoneal primary macrophages and mouse lung epithelial cells BNCC341334 was examined.

5×10⁵ cells per well were inoculated in a 24-well cell culture plate, and cultured in a 5% CO2 incubator at 37° C. until full confluence of the cells. The non-adherent cells were washed away with PBS, and the cells were added with medium with the final melatonin concentrations of 1, 2, 3 and 10 mg/mL sequentially. The melatonin-free DMSO solvent was used as the control group. After melatonin treatment for 12 h, the supernatant was collected for LDH test. For specific test steps, please refer to the LDH test kit as is known in the art. Each experiment was repeated three times, and each group was set in hexaplicate.

2×10⁵ mouse lung epithelial cells per well were inoculated into a 48-well microplate, and cultured in a 5% CO₂ incubator at 37° C. until full confluence of the cells. The non-adherent cells were washed away with 1×PBS, and the cells were treated with melatonin with the final concentrations of 1, 2, 3, 10, 20 and 40 mg/ml, while the control group was treated with the same volume of DMSO. After melatonin treatment for 30 min, 1MOI Pasteurella multocida was added into the culture medium for continue culture. After 12 h, the bacterial cells were collected, lysed by ultrasound at a low temperature, and then the enzyme activity of citrate synthase was analyzed. For specific test steps, please refer to the kit for detecting the enzyme activity of the citrate synthase. Each experiment was repeated three times, and each group was set in hexaplicate.

In additional studies, 1 mL of Klebsiella pneumoniae, Pseudomonas aeruginosa, Diplococcus pneumoniae and Streptococcus pneumoniae at the logarithmic growth stage was added into 100 mL of fresh and sterile Martin broth medium containing different concentrations of melatonin and citric acid (CA) (MT-1 mg/mL, MT-1 mg/mL-CA-1 mg/mL, MT-1 mg/mL-CA-2 mg/mL, MT-1 mg/mL-CA-3 mg/mL; MT-2 mg/mL, MT-2 mg/mL-CA-1 mg/mL, MT-2 mg/mL-CA-2 mg/mL, MT-2 mg/mL-CA-3 mg/mL; MT-3 mg/mL, MT-3 mg/mL-CA-1 mg/mL, MT-3 mg/mL-CA-2 mg/mL MT-3 mg/mL-CA-3 mg/mL). DMSO (that did not contain melatonin and citric acid) was used as a solvent control group, and bacterial cells were cultured in a constant-temperature shaker at 220 rpm and 37° C., and then 12 h later the growth curve of the cells was determined.

Further, 1 mL of Klebsiella pneumoniae, Pseudomonas aeruginosa, Diplococcus pneumoniae and Streptococcus pneumoniae at the logarithmic growth stage was added into 100 mL of fresh and sterile Martin broth liquid medium containing different concentrations of melatonin and citric acid (MT-1 mg/mL, MT-2 mg/mL, MT-3 mg/mL, CA-1 mg/mL, CA-2 mg/mL, CA-3 mg/mL, MT-1 mg/mL-CA-1 mg/mL, MT-2 mg/mL-CA-2 mg/mL, MT-3 mg/mL-CA-3 mg/mL) added was. Melatonin-free DMSO was used as the solvent control group, and the bacterial cells were cultured in a constant-temperature shaker at 37° C. and 220 rpm for 12 h. Thereafter, 1 mL of the bacterial solution was pipetted and centrifuged at 3000 r/min for 10 minutes. The bacterial cells were then collected and determined for citrate synthase activity according to the protocol of the instructions for the kit. Each experiment was repeated three times, and each group was set in triplicate.

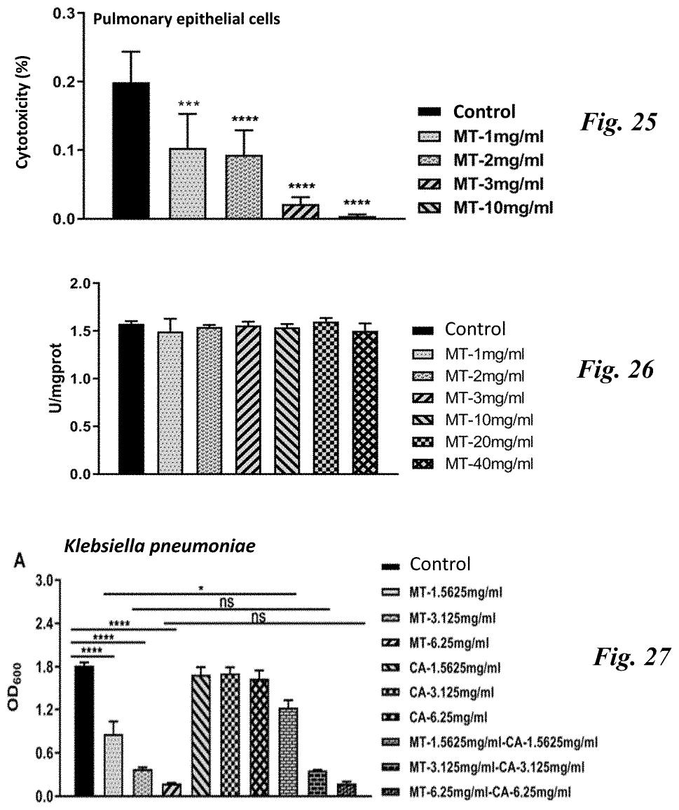

It can be seen in FIGS. 24 and 25 that no enhanced cytotoxicity was observed in mouse peritoneal primary macrophages and mouse lung epithelial cells after exposure to different concentrations of melatonin. On the contrary, mammalian cells treated with melatonin showed a higher survival rate after 24 hours of culture. Furthermore, melatonin up to 40 mg/mL did not affect the enzyme activity of citrate synthase in lung epithelial cells (see FIG. 26), which indicates melatonin is safe for the treatment of mammals.

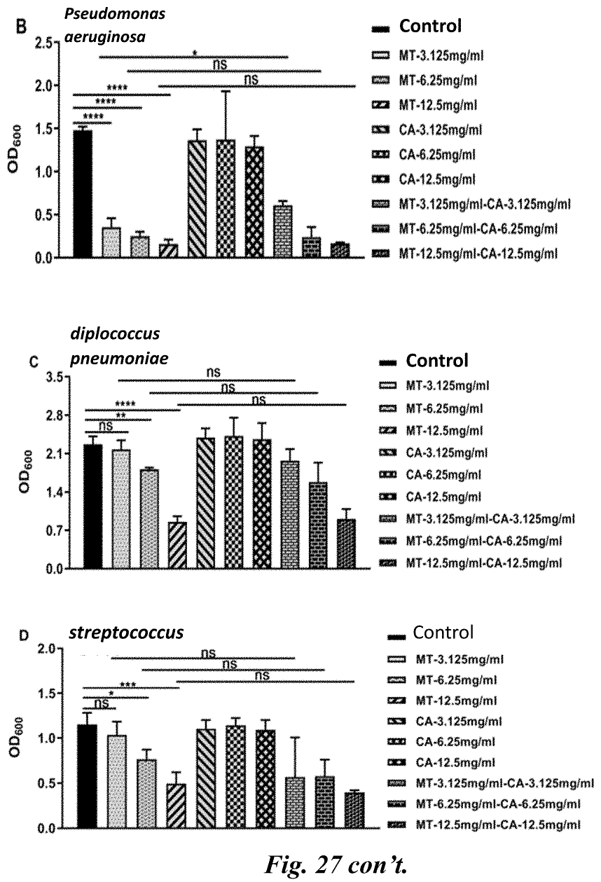

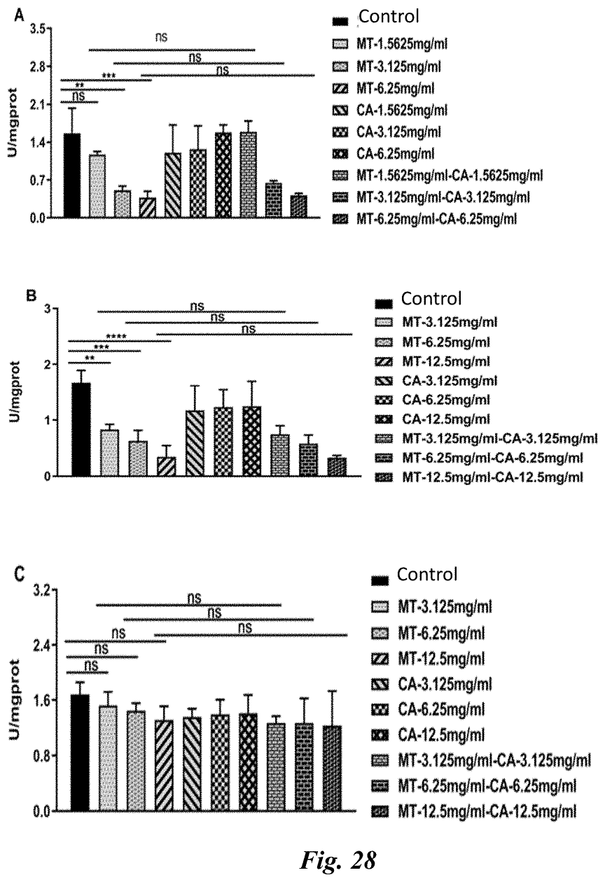

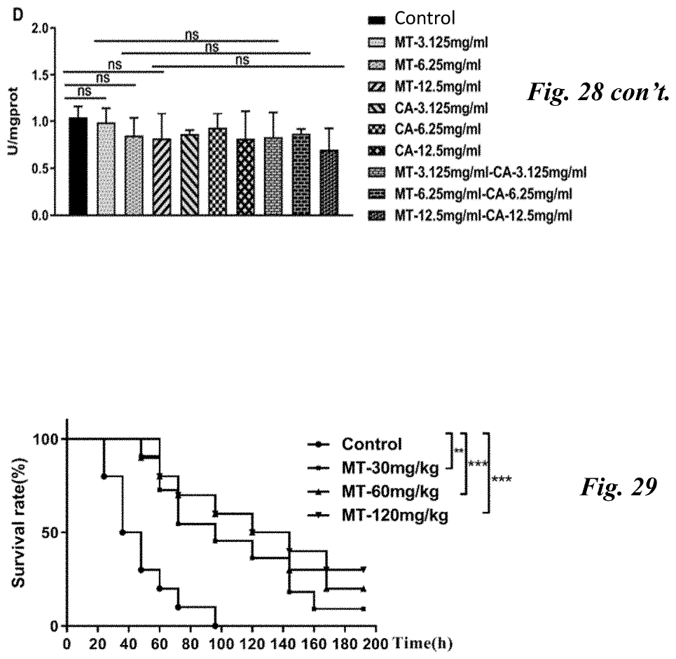

It can be seen from FIGS. 27 and 28 that, although melatonin exhibits antibacterial activity against both gram-negative bacteria (Klebsiella pneumoniae and Pseudomonas aeruginosa) and gram-positive bacteria (Streptococcus and Pneumococcus), melatonin significantly reduces the enzyme activity of citrate synthase in Klebsiella pneumoniae and Pseudomonas aeruginosa. Additionally, the exogenous addition of citric acid can reduce the bacteriostatic effect of melatonin on Klebsiella pneumoniae and Pseudomonas aeruginosa, while the enzyme activity of citrate synthase in Streptococcus and Pneumococcus was not influenced, even in the case of the exogenous addition of citric acid. In view of the above, the data indicates that melatonin exerts the antibacterial activity against gram-negative bacteria by specifically inhibiting the activity of citrate synthase of bacteria and reducing the synthesis of citric acid.

### Example 10

### Test of Prevention and Treatment of Bacterial Infection by Melatonin

In view of the remarkable in vitro bacteriostatic activity and novel bacteriostatic mechanism of melatonin, the in vivo preventive and therapeutic potential of melatonin was studied.

1. Test of Prevention of Pasteurella multocida Infection by Melatonin:

C57 mice purchased from the Third Military Medical University were randomly divided into four groups (10 mice in each group): a group of Pasteurella multocida+solvent control, a group of Pasteurella multocida+MT (30 mg/kg), a group of Pasteurella multocida+MT (60 mg/kg), and a group of Pasteurella multocida+MT (120 mg/kg). Before Pasteurella multocida infection, MT was injected intraperitoneally to the experimental group (injection once in the morning) for consecutive 7 days (P) or 6 days (P-1), while the control mice received the equal amount of DMSO. Thereafter, mice were infected by intraperitoneal injection of suspension of Pasteurella multocida (2.2∴10⁵ CFU). The survival rate of mice within 7 days was monitored and recorded. Blood samples were collected from mice, and the serum was stored at −20° C. for later ELISA test. Lung samples were collected and homogenized aseptically, and then the bacterial load was quantified by serial dilution, plate inoculation and CFU counting.

2. Test of Treatment of Pasteurella multocida Infection by Melatonin:

Mice were randomly divided into four groups (10 mice in each group) as described in #1, described above. In the experimental group, after infection with 2.2×10⁵ CFU of Pasteurella multocida through intraperitoneal injection, MT was injected intraperitoneally for four times, with the first injection 30 minutes after Pasteurella multocida infection, then the injection was performed once every 6 hours, for three times in total. Control mice were given an equal amount of DMSO at the same four time points. Subsequently, the survival rate of mice within 7 days was recorded. At the same time, mice were euthanized at 12 h, 16 h, 24 h and 32 h after infection to collect tissue and serum samples for later ELISA and bacterial count analysis.

3. Test of Colonization of Mouse Lung Bacteria Before and After Melatonin Treatment

In order to measure the colonization of mouse lung bacteria before and after melatonin treatment, the lung tissues of mice were collected 24 hours and 32 hours after infection. The tissues were homogenized under aseptic conditions, and the bacterial content was determined by continuous dilution of 10 times in saline. These different dilutions were coated on the Martin broth agar in triplicate and incubated in a constant-temperature incubator at 37° C. for 24 hours to count the bacterial CFU.

4. RT-PCR Analysis:

The mouse lung tissue samples were collected, and the total RNA of mouse lung tissues was extracted according to the instructions of the total RNA extraction kit (Invitrogen). According to the instructions of a TaKaRa reverse transcription kit, cDNA was prepared and reverse transcribed with the general process as follows:

1) Genomic DNA Elimination Reaction:

The reaction system was as follows:

| Reagents | Volume |
|---|---|
| 5x gDNA Eraser Buffer | 2.0 μL |
| gDNA Eraser | 1.0 μL |
| Total RNA | 2 μL |
| RNase Free dH₂O | 5 μL |
| Total | 10.0 μL |

The reaction procedure was 42° C. for 2 min, and stored at 4° C.

2) Reverse Transcription Reaction:

The reaction system was as follows:

| Reagents | Volume |
|---|---|
| Reaction solution from Step 1 | 10.0 μL |
| 5x PrimeScript Buffer 2 | 4.0 μL |
| PrimeScript RT Enzyme Mix I | 1.0 μL |
| RT Primer Mix | 1.0 μL |
| RNase Free dH₂O | 4.0 μL |
| Total | 20.0 μL |

The reaction procedure was 37° C. for 15 min, 85° C. for 5 s, and stored at 4° C.

3) qRT-PCR

The process of qRT-PCR was carried out with reference to previous studies, and (3-actin was used as a reference gene to normalize the transcription level of the target gene. The specific primer sequences used are shown in Table 2. The relative expression level of the gene was calculated according to the equation 2^{-(ΔΔCt)}, wherein ΔΔCt=(CT of the target gene−Ct of the internal reference) of the experimental group−(Ct of the target gene−Ct of the internal reference) of the control group.

TABLE 2
Primers of Inflammatory Cytokines

| | Accession | | Sequence |
|---|---|---|---|
| Gene | Number | Primer (5′→3′) | Length |
| Il-1 β | NM_008361.3 | F: ATGAAAGACGGCACACCCAC (SEQ ID NO. 1) | 175 |
| | | R: GCTTGTGCTCTGCTTGTGAG (SEQ ID NO. 2) | |
| Tnf-α | NM_013693.2 | F: AGGCACTCCCCCAAAAGAT (SEQ ID NO. 3) | 192 |
| | | R: TGAGGGTCTGGGCCATAGAA (SEQ ID NO. 4) | |
| Ifn-γ | NM_008337.4 | F: GCTTTGCAGCTCTTCCTCA (SEQ ID NO. 5) | 153 |
| | | R: CTTTTGCCAGTTCCTCCAG (SEQ ID NO. 6) | |
| Il-6 | NM_031168.1 | F: TGCAAGAGACTTCCATCCAGT (SEQ ID NO. 7) | 71 |
| | | R: GTGAAGTAGGGAAGGCCG (SEQ ID NO. 8) | |
| Il-4 | NM_021283.2 | F: CCATATCCACGGATGCGACA (SEQ ID NO. 9) | 166 |
| | | R: AAGCACCTTGGAAGCCCTAC (SEQ ID NO.10) | |
| Il-10 | NM_010548.2 | F: GGCGCTGTCATCGATTTCTC (SEQ ID NO. 11) | 103 |
| | | R: ATGGCCTTGTAGACACCTTGG (SEQ ID | |
| | | NO. 12) | |
| Beta-actin | NM_007393.3 | F: GTCCACCTTCCAGCAGATGT (SEQ ID NO. 13) | 117 |
| | | R: GAAAGGGTGTAAAACGCAGC (SEQ ID NO. | |
| | | 14) | |

5. Enzyme Linked Immunosorbent Assay (ELISA):

ELISA samples were divided into serum samples and lung tissue homogenate samples. Among them, the process of collecting serum was collecting mouse whole blood from the eyeball, storing overnight at 4° C., centrifuging at 5000 rpm for 10 min, collecting the supernatant and storing it at −20° C. for later use. While the preparation process of the mouse lung homogenate was collecting lung tissues, adding 2 mL sterile normal saline to prepare a homogenate, then repeatedly freezing and thawing the homogenate in liquid nitrogen (5 min) and ice-water bath for 3 times, then centrifuging at 4° C. and 12000 rpm for 10 min, and collecting the supernatant for later use. According to the instructions of the kit, the serum and the supernatant of lung tissue homogenate as prepared were detected for IL-10, IL-4, IL-6, IL-10, TNF-α and IFN-γ (eBioscience).

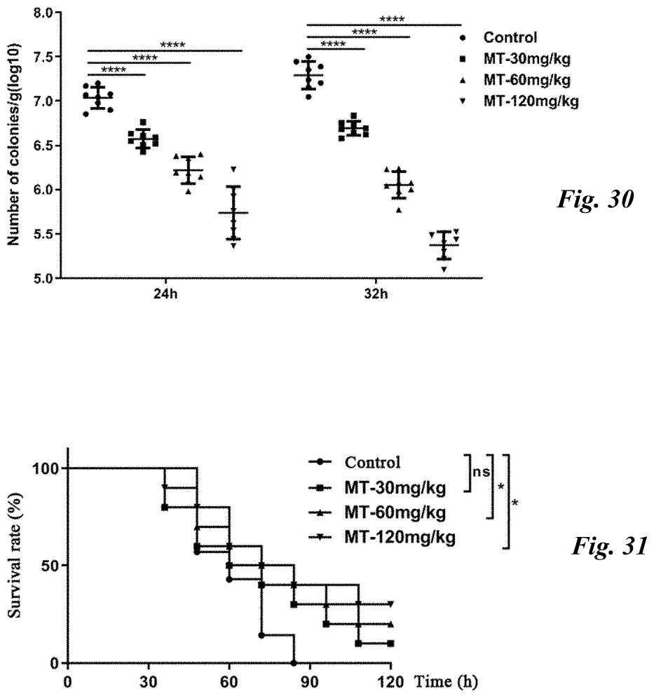

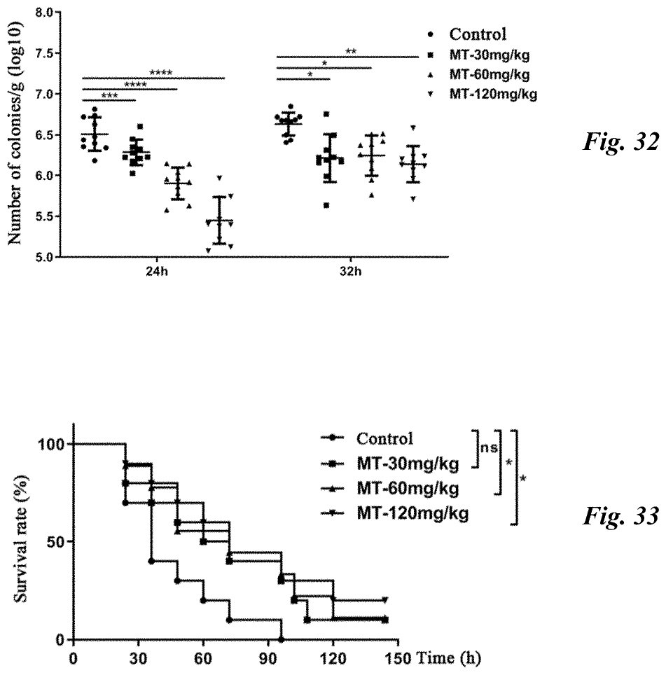

The data illustrated in FIGS. 29-34 show that, as compared with the DMSO group (all mice died within 100 hours of infection), the survival rates of mice in the prevention and treatment groups were significantly improved and the bacterial colonization in the lung tissues of mice 24 hours and 32 hours after infection was significantly reduced. At the dose of 120 mg/kg, the bacterial colonization amount (CFU/g) was decreased by about 2-log 10.

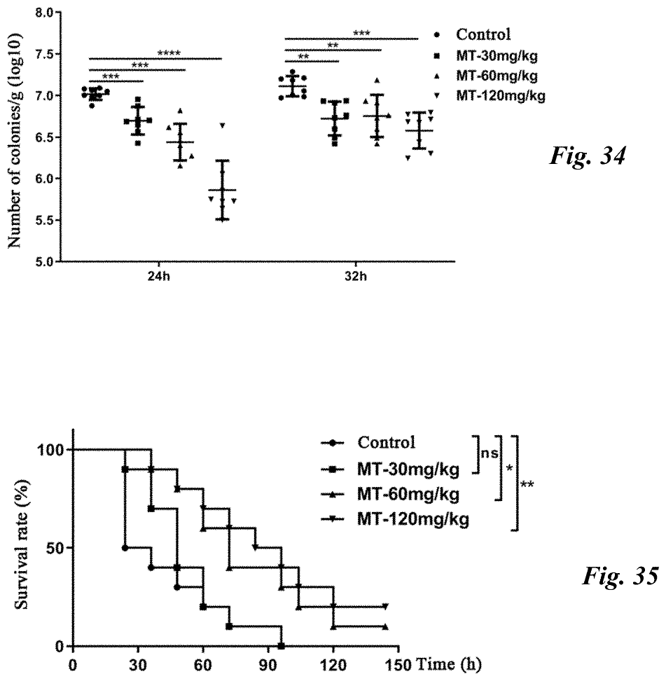

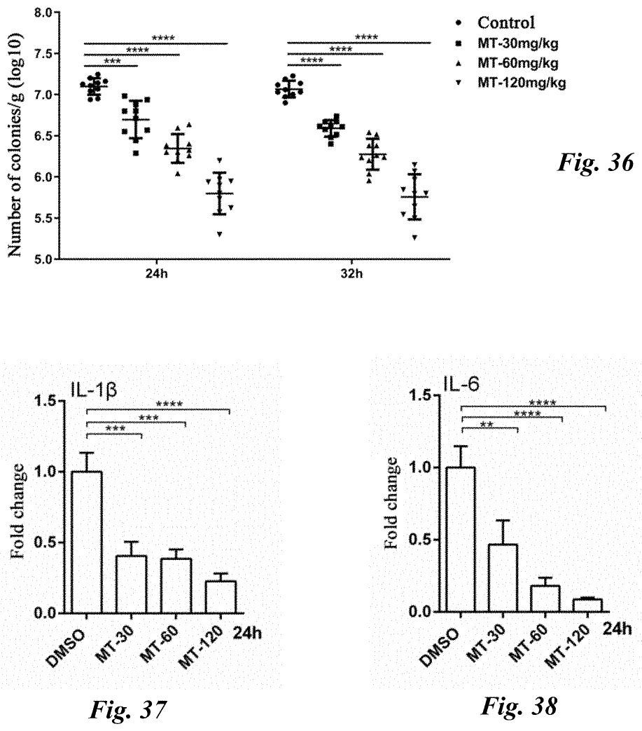

Furthermore, before Pasteurella multocida infection, all mice were intraperitoneally injected with melatonin for consecutive 7 days to test the preventive effect of melatonin against bacterial infection. The results shown in FIGS. 35 and 36 show that when the preventive dose of melatonin was increased to 60 mg/kg or above, the survival rate of mice was significantly improved, and the bacterial count in the lung was significantly reduced compared with those of the control group. Even if melatonin was removed 24 hours before Pasteurella multocida infection, melatonin still had a significant preventive effect on the bacterial infection (see FIGS. 33 and 34).

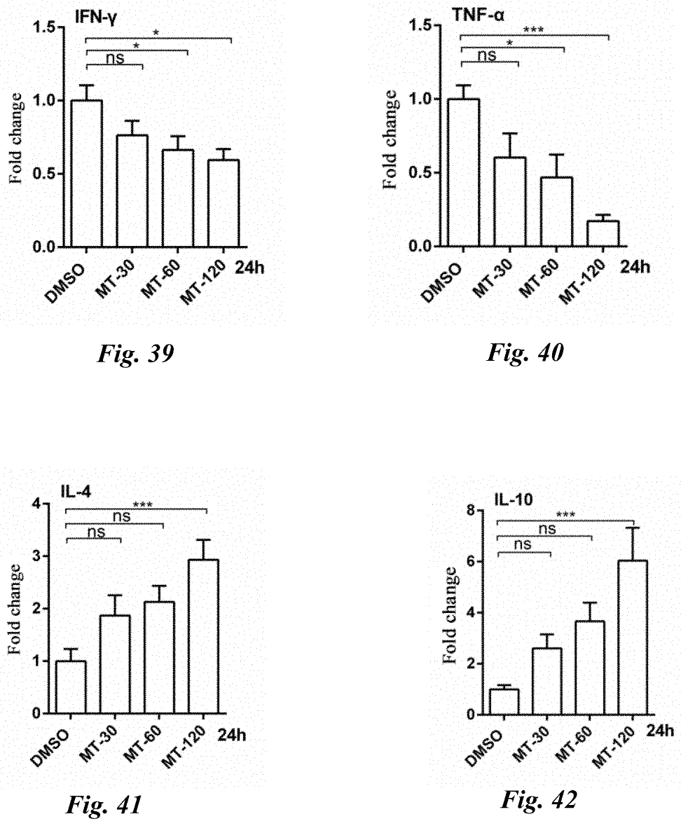

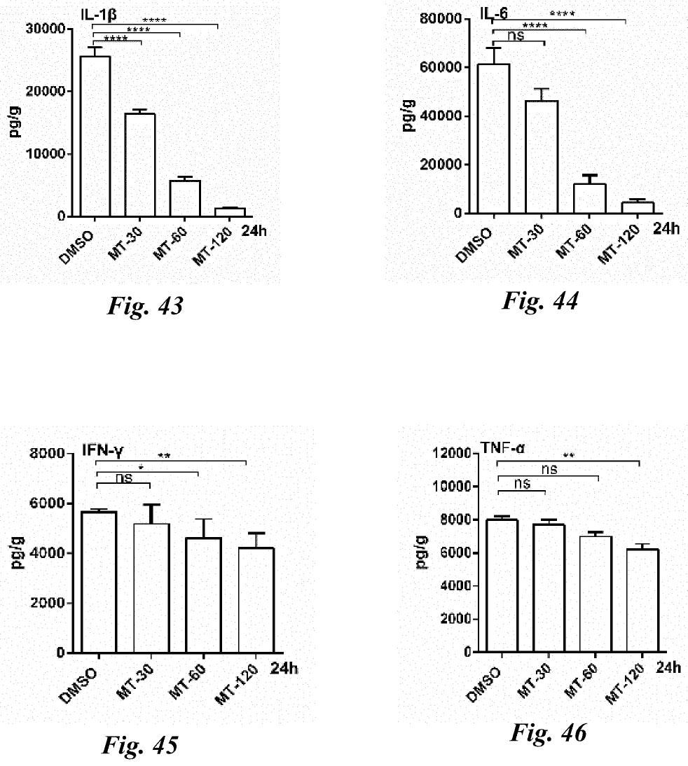

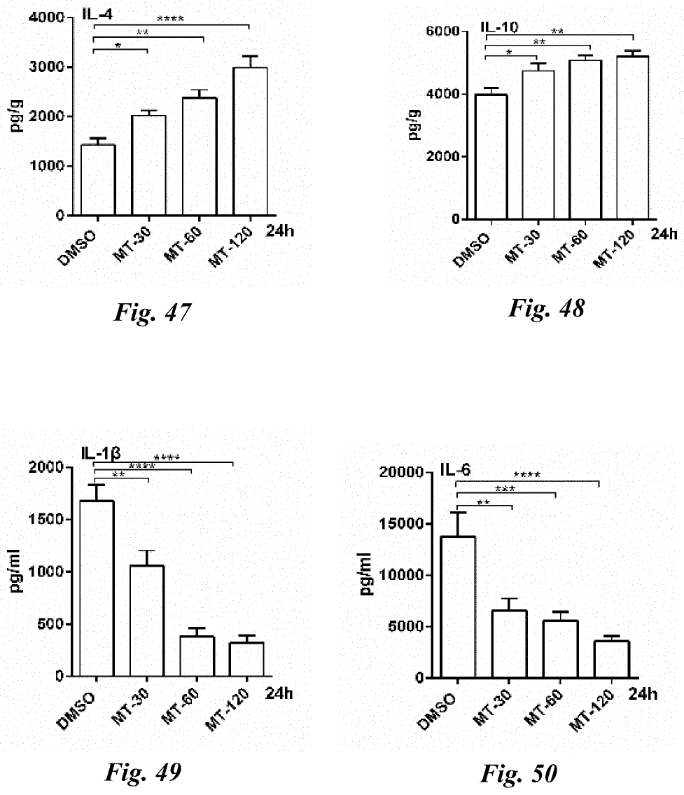

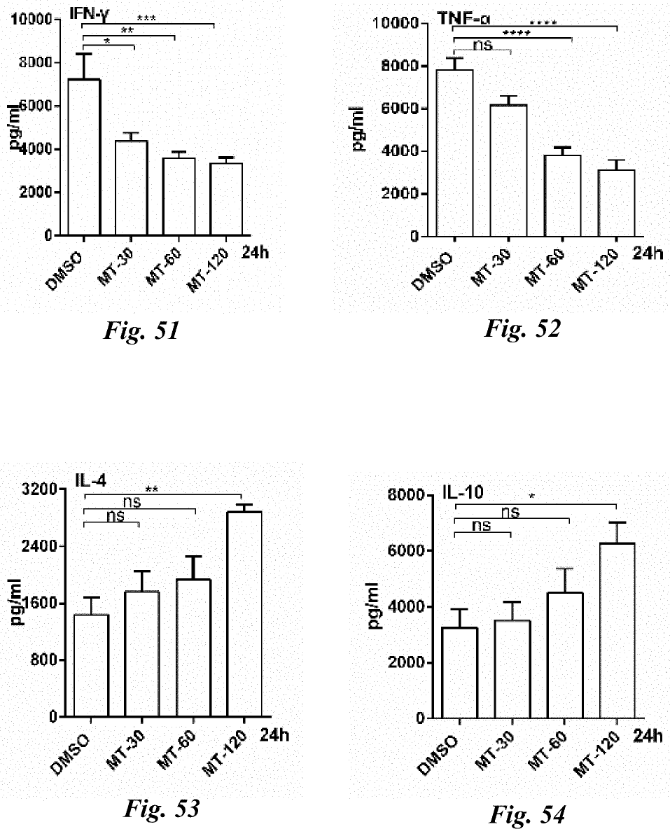

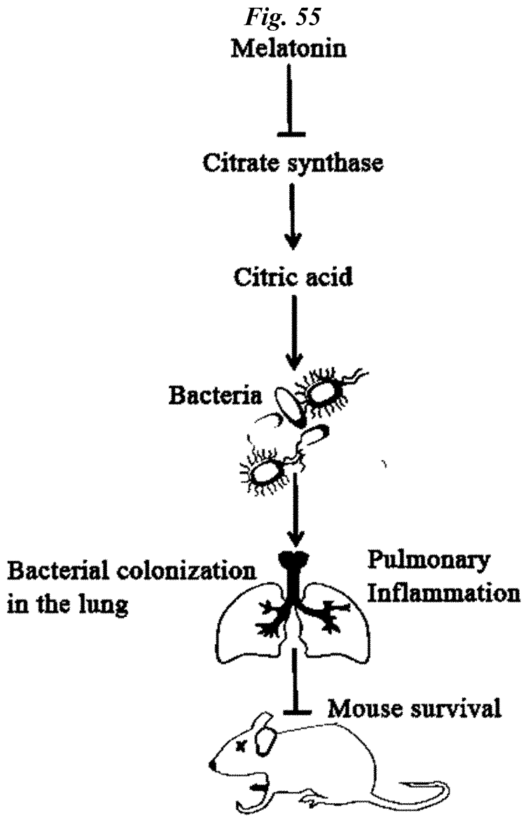

Finally, melatonin was injected intraperitoneally for four times respectively at 30 minutes, 6 hours, 12 hours and 18 hours after the bacterial infection to explore the therapeutic effect of melatonin on Pasteurella multocida infection. The results show that it had obvious protective effects, including improving the survival rate and reducing bacterial colonization in mouse lungs (see FIGS. 35 and 36). Pulmonary inflammation was the most important pathological feature of Pasteurella multocida infection. Interestingly, 24 hours after infection, it was found through RT-PCR and ELISA analysis that, melatonin significantly reduced the expression of IL-10, IL-6, IFN-γ and TNF-α, but promoted the transcription and expression of IL-4 and IL-10 (see FIGS. 37-54). These data prove that melatonin reduced the inflammatory response by inhibiting pro-inflammatory factors and activating anti-inflammation. Generally speaking, these data indicate that melatonin not only has a direct antibacterial effect, but also controls bacterial infection through various ways of action, including reducing the inflammatory response and bacterial virulence in vivo. In summary, these data indicate that treatment with melatonin (and compositions comprising melatonin) can effectively prevent and treat Pasteurella multocida diseases.

The above description describes preferred embodiments of the present invention and is not intended to be limiting. It should be pointed out that, for those of ordinary skills in the art, several improvements and modifications can be made without departing from the principle of the present invention. These improvements and modifications should also be considered as falling into the claimed scope of the present invention.

What is claimed is:

1. A method of preventing or treating a disease state in a subject, the method comprising the steps of administering to the subject an amount of a medicament comprising melatonin as an active ingredient, the medicament for inhibiting and/or killing a bacterium related to the disease state.

2. The method according to claim 1, wherein the bacterium is a gram-positive bacterium or a gram-negative bacterium.

3. The method according to claim 2, wherein the gram-positive bacterium comprises Bacillus cereus, Streptococcus pneumoniae, Pneumococcus, Listeria monocytogenes, Lactobacillus johnsonii or Staphylococcus aureus.

4. The method according to claim 2, wherein the gram-negative bacterium comprises Pasteurella multocida, Klebsiella pneumoniae, avian pathogenic Escherichia coli, Enterobacter sakazakii, Pseudomonas aeruginosa, Acinetobacter baumannii, Salmonella enteritidis, bovine meningitic Escherichia coli or Mannheimia haemolytica.

5. The method according to claim 1, wherein the melatonin damages the cell membrane of a bacterium.

6. The method according to claim 1, wherein the disease state is a bacterial infection disease.

7. The method according to claim 1, wherein the disease state comprises pulmonary inflammation.

8. The method according to claim 1, wherein the medicament comprises melatonin in a concentration of 1.5625 mg/mL.

9. The method according to claim 1, wherein the medicament comprises a dosage of melatonin in an amount of 60 mg/kg or higher.

10. A bacterial metabolic blocker composition comprising melatonin.

11. The composition according to claim 10, wherein the bacterial metabolic blocker is formulated to block pathways of bacterial metabolism comprising pyruvate metabolism, citric acid cycle and tryptophan metabolism.

12. A pharmaceutical composition comprising melatonin as an active ingredient and a pharmaceutically acceptable carrier.

13. The pharmaceutical composition of claim 12, wherein the melatonin has a content of 1.5625 mg/mL in the pharmaceutical composition.

14. The pharmaceutical composition of claim 12, wherein the pharmaceutically acceptable carrier comprises one or more of a diluent, a colorant, a sweetener, a coating agent, an adhesive, an absorbent, a disintegrating agent, a releasing agent, a dispersing agent, a wetting agent, a cosolvent, a buffer, and a surfactant.

15. The pharmaceutical composition of claim 12 formulated for dosage as a solid, a liquid or a gas.


SEQUENCE LISTING (14 sequences)

SEQ ID NO: 1 - DNA, 20 bp, artificial sequence
  IL-1Beta Upstream Primer
atgaaagacggcacacccac

SEQ ID NO: 2 - DNA, 20 bp, artificial sequence
  IL-1Beta Downstream Primer
gcttgtgctctgcttgtgag

SEQ ID NO: 3 - DNA, 19 bp, artificial sequence
  TNF-Alpha Upstream Primer
aggcactcccccaaaagat

SEQ ID NO: 4 - DNA, 20 bp, artificial sequence
  TNF-Alpha Downstream Primer
tgagggtctgggccatagaa

SEQ ID NO: 5 - DNA, 19 bp, artificial sequence
  IFN-Gamma Upstream Primer
gctttgcagctcttcctca

SEQ ID NO: 6 - DNA, 19 bp, artificial sequence
  IFN-Gamma Downstream Primer
cttttgccagttcctccag

SEQ ID NO: 7 - DNA, 21 bp, artificial sequence
  IL-6 Upstream Primer
tgcaagagacttccatccagt

SEQ ID NO: 8 - DNA, 18 bp, artificial sequence
  IL-6 Downstream Primer
gtgaagtagggaaggccg

SEQ ID NO: 9 - DNA, 20 bp, artificial sequence
  IL-4 Upstream Primer
ccatatccacggatgcgaca

SEQ ID NO: 10 - DNA, 20 bp, artificial sequence
  IL-4 Downstream Primer
aagcaccttggaagccctac

SEQ ID NO: 11 - DNA, 20 bp, artificial sequence
  IL-10 Upstream Primer
ggcgctgtcatcgatttctc

SEQ ID NO: 12 - DNA, 21 bp, artificial sequence
  IL-10 Downstream Primer
atggccttgtagacaccttgg

SEQ ID NO: 13 - DNA, 20 bp, artificial sequence
  Beta-actin Upstream Primer
gtccaccttccagcagatgt

SEQ ID NO: 14 - DNA, 20 bp, artificial sequence
  Beta-actin Downstream Primer
gaaagggtgtaaaacgcagc